(19) United States
(12) Patent Application Publication
(10) Pub. No.: US 2006/0064052 A1
(43) Pub. Date: Mar. 23, 2006

(54) Liquid delivering device

(75) Inventors: Xuan Xiong Zhang, Shanghai (CN); Yuan Xu, Singapore (SG); Yen Peng Kong, Singapore (SG); Kui Yao, Singapore (SG); Xu Jiang He, Singapore (SG)

Correspondence Address: Parsons Behle & Latimer, 201 South Maine Street, Suite 1800, Salt Lake City, UT, 84111-2218, US

(73) Assignee: Agency for Science, Technology & Research, Centros (SG)

(21) Appl. No.: 10/488,338

(22) PCT Filed: Aug. 7, 2002
(86) PCT No.: PCT/SG02/00180

§ 371 Date: Nov. 5, 2004

(30) Foreign Application Priority Data
Aug. 31, 2001 (SG) 200105332-1

(51) Int. Cl. A61N 1/30

(52) U.S. Cl. 604/20

(57) ABSTRACT

An electrochemically actuated liquid delivering device which is capable of releasing specific volumes of liquid at desired flow rates. The device includes a sealed electrolytic chamber which is adapted to contain an electrolyte and has at least one pair of electrodes which are at least partially in contact with the electrolyte. It also has a liquid chamber formed of a rigid material which is housed at least partially within the electrolytic chamber and is adapted to contain a liquid. It has pressure transfer means separating the electrolytic chamber from the liquid chamber and administering means which is adapted to release the liquid from the liquid chamber under the influence of pressure applied to the liquid chamber. The device is arranged so that when current is passed through the electrolyte via the electrodes, gas is generated from the electrolyte thereby increasing pressure inside the electrolytic chamber. That increase in pressure is transferred to the liquid chamber via the pressure transfer means and a determinable amount of the liquid is released from the liquid chamber via the administering means. The device has particular application in liquid drug delivery systems such as insulin infusion.

### TECHNICAL FIELD

The present invention relates to liquid delivering devices and, in particular, relates to a liquid delivering device powered by electrolysis and which is capable of releasing specific volumes of liquid at desired flow rates.

The invention is particularly suitable for administering liquids containing drugs to subjects and will be described with reference to this specification. However, it will be appreciated that the invention is not limited to this particular field of use.

### BACKGROUND ART

There is a need in a number of medical applications, such as drug delivery systems, for a device that is able to release liquid in small volumes and at precise or desired rates. One such example is in insulin infusion. Type I diabetics (and about 30% of Type II diabetics) need a regular supply of insulin in their blood stream to aid the absorption of glucose. In an ideal situation, the insulin supply should maintain the insulin in the blood at a level which corresponds with the daily digestive cycle of a normal human. If the insulin is supplied at this level, the diabetic is able to lead a normal life. Traditional methods of injecting insulin via a syringe do not achieve this goal due to the intermittent nature of the insulin supply.

A new therapy, known as “continuous insulin infusion” has been suggested in which a portable delivery device is attached to or implanted inside the patient's body. The device would preferably be able to deliver an exact amount of insulin (such as a few nano litres per second) into the patient's blood, according to the patient's blood glucose concentration. In order to produce such an infusion system, a precision liquid delivery device is needed.

Some of the currently available commercial devices have a number of problems. Some devices have been too large to be comfortably fitted to a patient. Others have been too expensive to allow the devices to be disposable. Some devices have difficulty in preventing the access of gas bubbles to the drug liquid, whilst the device is in operation. Injecting liquids containing gas into patients can have disastrous effects. Other devices have a two-way action which allows the drug liquid to be sucked into the device and then released from the device. This two-way action has the disadvantage of allowing contaminants and gases to mix with the drug liquid. A further disadvantage of known prototypes is that they can only be operated in a limited number of orientations, thereby restricting their effectiveness.

One advantage of the present invention is that it overcomes, or ameliorates at least some of the disadvantages of the prior art, and provides a useful alternative.

Although the present invention is particularly suitable for use in insulin infusion systems, it may also be used for any medical, or other, applications which require the precise delivery of a drug or other liquid. Some non-limiting examples include the on-demand delivery of hypertension drugs and hormones, the delivery of liquid drugs to prematurely born babies in intensive care situations, and the like.

### DISCLOSURE OF INVENTION

In a first aspect, the present invention provides a liquid delivering device comprising:

(a) a sealed electrolytic chamber adapted to contain an electrolyte and having at least one pair of electrodes at least partially inside the chamber;

(b) a liquid chamber housed at least partially within the electrolytic chamber and adapted to contain a liquid, said liquid chamber comprising a base and a sidewall each formed of a substantially rigid material;

(c) pressure transfer means separating the electrolytic chamber from the liquid chamber; and

(d) administering means adapted to release the liquid from the liquid chamber under influence of pressure applied to the liquid chamber

whereby when the electrolytic chamber contains an electrolyte, the liquid chamber contains a liquid and a current is passed through the electrolyte via the electrodes:

(i) gas is generated from the electrolyte thereby increasing pressure inside the electrolytic chamber;
(ii) that increase in pressure is transferred to the liquid chamber via the pressure transfer means; and
(iii) a determinable amount of the liquid is released from the liquid chamber via the administering means.

Preferably, the pressure transfer means comprises one or more of:

(a) a deformable membrane;

(b) a bellow;

(c) a piston;

(d) a diaphragm; and

(e) a bladder.

When the pressure transfer means comprises a deformable membrane, the deformable membrane is preferably at least partially made from one or more of:

(a) hyper-elastic materials;

(b) materials with high elasticity; and

(c) materials with high plasticity which resist permeation by one or more of:

(a) gas;

(b) liquid;

(c) semi solids; and

(d) gels.

and the deformable membrane is at least partially joined to an upper edge of the liquid chamber.

Preferably, the electrolytic and liquid chambers are substantially co-axial, are substantially cylindrical in shape, the liquid chamber is contained wholly within the electrolytic chamber and the electrolytic and liquid chambers share a common base.

Preferably, the administering means comprises one or more of:

(a) one or more one-way valves;

(b) one or more needles; and

(c) one or more nozzle holes.

When the administering means comprises one or more needles, they preferably comprise one or more micro injection needles.

When the administering means comprises a one-way valve, the one-way valve preferably comprises a one-way check valve comprising:

(a) a first wafer having an opening; and

(b) a second wafer having a deformable flange

wherein the first and second wafers are at least partially bonded to one another so that the deformable flange covers the opening. More preferably, the deformable flange is biased towards a closed position in which it covers the opening. Even more preferably, when the liquid in the liquid chamber reaches a threshold pressure the deformable flange deforms outwardly, uncovering the opening and releasing a determinable amount of the liquid from the liquid chamber.

In one form one or both of the first and second wafers are monolithic silicon wafers manufactured using micromachining techniques.

Preferably, the administering means is formed in a base of the liquid chamber.

Preferably, the electrolytic chamber further includes a sensor adapted to sense one or more of the:

(a) internal pressure; and

(b) internal temperature of the electrolytic chamber. More preferably, the sensor is in communication with a current controller and is adapted to provide information to the current controller regarding one or more of the:

(a) internal pressure; and

(b) internal temperature of the electrolytic chamber.

In one form the device is adapted to release the liquid at a flow rate of between 1 and 1000 micro litres per second.

In another form, the device is adapted to release the liquid at a flow rate of between 1 and 1000 nano litres per second.

In a further form, the device is adapted to release the liquid at a flow rate of between 1 and 1000 pico litres per second.

Preferably, at least part of the device is adapted to be used only once. More preferably, the liquid chamber is adapted to be filled with the liquid only once. Even more preferably, the electrolytic chamber is adapted to be filled with the electrolyte only once.

In one form, the electrolytic chamber is adapted to be removably attachable to the base. Preferably the device further includes a sealing component located between the electrolytic chamber and the base when they are attached. The device may also include a fastening mechanism adapted to removably attach the electrolytic chamber to the base.

Preferably, the electrolytic chamber comprises:

(a) an electrolytic chamber housing defining an electrolytic chamber cavity; and

(b) at least one pair of electrodes extending at least partially into the electrolytic chamber cavity.

Preferably, the at least one pair of electrodes are spaced at regular intervals within the electrolytic chamber cavity.

In one form, the at least one pair of electrodes comprises two pairs of electrodes spaced at regular intervals within the electrolytic chamber cavity.

In another form, the at least one pair of electrodes comprises three pairs of electrodes spaced at regular intervals within the electrolytic chamber cavity.

In a further form, the at least one pair of electrodes comprises four pairs of electrodes spaced at regular intervals within the electrolytic chamber cavity.

Preferably, the electrodes are at least partially made from one or more of:

(a) metals and alloys;

(b) conductive metal oxides;

(c) conductive metal halides;

(d) conductive silicides;

(e) conductive borides;

(f) conductive carbides;

(g) conductive nitrides;

(h) multi-layer conductors; and

(i) other conductive materials.

Preferably, the electrolytic chamber housing is at least partially made from substantially rigid materials.

Preferably, the substantially rigid material of which the electrolytic chamber and the liquid chamber are formed includes materials manufactured using one or more of the following techniques:

(a) microfabrication techniques;

(b) injection moulding; and

(c) mechanical machining.

Preferably, the device described above is at least partially made from one or more of:

(a) polymeric materials;

(b) ceramic materials;

(c) metals; and

(d) metal alloys.

In one form, the liquid is a drug liquid such as insulin.

Preferably, the gas liberated from the electrolyte is non-toxic.

In a second aspect, the present invention provides a method of administering a liquid to a subject, the method comprising the steps of:

(a) providing a liquid delivering device comprising:
(i) a sealed electrolytic chamber containing an electrolyte and having at least one pair of electrodes at least partially in contact with the electrolyte;
(ii) a liquid chamber housed at least partially within the electrolytic chamber containing a liquid, said liquid chamber comprising a base and a sidewall each formed of a substantially rigid material;
(iii) pressure transfer means separating the electrolytic chamber from the liquid chamber; and
(iv) administering means adapted to release the liquid from the liquid chamber under influence of pressure applied to the liquid chamber

(b) connecting the administering means to the subject;

(c) passing a current through the electrolyte via the electrodes causing:
(i) gas to be generated from the electrolyte thereby increasing pressure inside the electrolytic chamber;
(ii) that increase in pressure to be transferred to the liquid chamber via the pressure transfer means; and
(iii) a determinable amount of the liquid to be released from the liquid chamber via the administering means.

In one form, the administering means comprises one or more injection needles and the step of connecting the administering means to the subject comprises inserting at least part of the one or more needles directly into the subject.

In another form, the administering means comprises one or more of:

(a) a one-way valve; and

(b) a nozzle and the step of connecting the administering means to the subject comprises:

(a) connecting a first end of a connecting tube to the one-way valve or nozzle; and

(b) connecting a second end of the connecting tube to the subject.

In a third aspect, the present invention provides a method of manufacturing a liquid delivering device, the method comprising the steps of:

(a) providing a substantially planar base having an outlet therein, the outlet adapted to allow selective passage of a liquid therethrough;

(b) providing a liquid chamber comprising a substantially rigid and substantially tubular housing having a first open end and a second open end;

(c) bonding the first open end of the liquid chamber housing to the base over the outlet;

(d) providing an electrolytic chamber comprising:
(i) a substantially tubular housing of a greater diameter and length than the liquid chamber housing and having a first open end and a second open end; and
(ii) at least one pair of electrodes at least partially inside the housing;

(e) bonding the first open end of the electrolytic chamber housing to the base over the liquid chamber so as to house the liquid chamber;

(f) providing a substantially planar deformable membrane having an inner section and an outer section;

(g) bonding the outer section of the membrane to the second open end of the liquid chamber housing;

(h) providing a substantially planar cover having an inner section and an outer section; and

(i) bonding the outer section of the cover to the second end of the liquid chamber housing.

Preferably the liquid chamber comprises a base and a sidewall each formed of a substantially rigid material. The electrolytic chamber is also preferably formed of a rigid material.

Preferably, the method includes the additional steps of:

(j) filling the liquid chamber with a liquid; and

(k) at least partially filling the electrolytic chamber housing with an electrolyte.

In one form, the step of (j) filling the liquid chamber with a liquid comprises the steps of:

(a) providing a one-way liquid inserting valve in the base, the valve adapted to provide one way liquid access to the inside of the liquid chamber; and

(b) filling the liquid chamber with a liquid via the valve.

In another form, the step of (j) filling the liquid chamber with a liquid comprises the steps of:

(a) providing a liquid inserting hole in the base;

(b) filling the liquid chamber with a liquid via the hole; and

(c) sealing the hole thereby sealing the liquid in the liquid chamber.

In one form, the step of (k) at least partially filling said electrolytic chamber housing with an electrolyte comprises the steps of:

(a) providing a one-way electrolyte inserting valve in the cover, the valve adapted to provide one-way electrolyte access to the inside of the electrolytic chamber; and

(b) at least partially filling the electrolytic chamber with an electrolyte via the valve.

In another form, the step of (k) at least partially filling said electrolytic chamber housing with an electrolyte comprises the steps of:

(a) providing an electrolyte inserting hole in the cover;

(b) filling the electrolytic chamber with an electrolyte via the hole; and

(c) sealing the hole thereby sealing the electrolyte in the electrolytic chamber.

Alternatively, step (d) is replaced by the step of:

(d1) providing an electrolytic chamber comprising a substantially tubular housing of a greater diameter and length than the liquid chamber housing and having a first open end and a second open end; and step (h) is replaced by the step of

(h1) providing a substantially planar cover having an inner section and an outer section and at least one pair of electrodes extending at least partially therefrom.

Preferably, the outlet comprises one or more of:

(a) one or more one-way valves;

(b) one or more needles; and

(c) one or more nozzle holes.

When the outlet comprises one or more needles, they preferably comprise one or more micro injection needles.

When the outlet comprises a one-way valve, it preferably comprises a one-way check valve comprising:

(a) a first wafer having an opening; and

(b) a second wafer having a deformable flange

wherein the first and second wafers are at least partially bonded to one another so that the deformable flange covers the opening. More preferably, the deformable flange is biased towards a closed position in which it covers the opening.

Preferably, when the liquid in the liquid chamber reaches a threshold pressure the deformable flange deforms outwardly, uncovering the opening and releasing a determinable amount of the liquid from the liquid chamber.

In one form one or both of the first and second wafers are monolithic silicon wafers manufactured using micromachining techniques.

In one form the wafers constitute the base.

Preferably, the electrolytic and liquid chambers are substantially co-axial, are substantially cylindrical in shape, the liquid chamber is contained wholly within the electrolytic chamber and the electrolytic chamber housing is at least partially made from substantially rigid materials.

Preferably, the liquid chamber housing is also at least partially made from substantially rigid materials.

Preferably, the substantially rigid materials include materials manufactured using one or more of the following techniques:

(a) microfabrication techniques;

(b) injection moulding; and

(c) mechanical machining.

In one form the substantially rigid materials include glass.

In another form, the substantially rigid materials include:

(a) polymeric materials;

(b) ceramic materials; and

(c) a metal or an alloy.

In one form the liquid is a drug liquid such as insulin.

Preferably, the deformable membrane is at least partially made from one or more of:

(a) hyper-elastic materials;

(b) materials with high elasticity; and

(c) materials with high plasticity which resist permeation by one or more of:

(a) gas;

(b) liquid;

(c) semi solids; and

(d) gels.

Preferably, the cover further includes a sensor adapted to sense one or more of the:

(a) internal pressure; and

(b) internal temperature

of the electrolytic chamber, is in communication with a current controller and is adapted to provide information to the current controller regarding one or more of the:

(a) internal pressure; and

(b) internal temperature

of the electrolytic chamber.

Preferably, the step of:

(c) bonding the first open end of the liquid chamber housing to the base over the outlet

comprises anodically bonding the liquid chamber housing to the base.

Preferably, the step of:

(e) bonding the first open end of the electrolytic chamber housing to the base over the liquid chamber so as to house the liquid chamber

comprises anodically bonding the electrolytic chamber housing to the base.

Preferably, the step of:

(j) filling the liquid chamber with a liquid

includes the preliminary or subsequent additional step of de-gassing the liquid.

Preferably, the at least one pair of electrodes are spaced at regular intervals around the inner wall of the electrolytic chamber housing.

In one form, the at least one pair of electrodes comprises two pairs of electrodes spaced at regular intervals around the inner wall of the electrolytic chamber housing.

In another form, the at least one pair of electrodes comprises three pairs of electrodes spaced at regular intervals around the inner wall of the electrolytic chamber housing.

In a further form, the at least one pair of electrodes comprises four pairs of electrodes spaced at regular intervals around the inner wall of the electrolytic chamber housing.

Preferably, the electrodes are at least partially made from one or more of:

(a) metals and alloys;

(b) conductive metal oxides;

(c) conductive metal halides;

(d) conductive silicides;

(e) conductive borides;

(f) conductive carbides;

(g) conductive nitrides;

(h) multi-layer conductors; and

(i) other conductive materials.

In one form the device is adapted to release the liquid at a flow rate of between 1 and 1000 micro litres per second.

In another form the device is adapted to release the liquid at a flow rate of between 1 and 1000 nano litres per second.

In a further form the device is adapted to release the liquid at a flow rate of between 1 and 1000 pico litres per second.

In one form the liquid chamber is adapted to be filled with the liquid only once and the electrolytic chamber is adapted to be filled with the electrolyte only once.

In a fourth aspect, the present invention provides a method of manufacturing a liquid delivering device, the method comprising the steps of:

(a) providing a substantially planar base having an outlet therein, the outlet adapted to allow selective passage of a liquid therethrough;

(b) providing a liquid chamber comprising a substantially rigid and substantially tubular housing having a first open end and a second open end;

(c) bonding the first open end of the liquid chamber housing to the base over the outlet;

(d) providing a substantially planar deformable membrane having an inner section and an outer section;

(e) bonding the outer section of the membrane to the second end of the liquid chamber housing;

(f) providing an electrolytic chamber comprising:
(i) a substantially tubular housing of a greater diameter and length than the liquid chamber housing and having an open end and a sealed end; and
(ii) at least one pair of electrodes at least partially inside the housing;

(g) inverting said electrolytic chamber housing so that its open end faces upwards;

(h) at least partially filling said electrolytic chamber housing with an electrolyte;

(i) inverting said liquid chamber so that its second end faces downwards;

(j) inserting the liquid chamber into the electrolytic chamber until the base comes into direct or indirect contact with the open end of the electrolytic chamber; and

(k) removably fastening the base to the electrolytic chamber using the fastening device.

Preferably, the method includes the additional step of filling the liquid chamber with a liquid.

In one form, the step of filling the liquid chamber with a liquid comprises the steps of:

(a) providing a one-way liquid inserting valve in the base, the valve adapted to provide one way liquid access to the inside of the liquid chamber; and

(b) filling the liquid chamber with a liquid via the valve.

In another form, the step of filling the liquid chamber with a liquid comprises the steps of:

(a) providing a liquid inserting hole in the base;

(b) filling the liquid chamber with a liquid via the hole; and

(c) sealing the hole thereby sealing the liquid in the liquid chamber.

In a fifth aspect, the present invention provides a method of assembling a liquid delivering device comprising:

(a) a substantially planar base having an outlet therein, the outlet adapted to allow selective passage of a liquid therethrough;

(b) a liquid chamber comprising a substantially rigid and substantially tubular housing having a first end sealed by a substantially planar deformable membrane, a second end bonded to the base over the outlet, and being filled with a liquid; and

(c) a separate electrolytic chamber comprising:
(i) a substantially tubular housing of a greater diameter and length than the liquid chamber housing and having an open end and a sealed end; and
(ii) at least one pair of electrodes at least partially inside the housing; the method comprising the steps of:

(a) inverting the electrolytic chamber housing so that its open end faces upwards;

(b) at least partially filling the electrolytic chamber housing with an electrolyte;

(c) inverting the liquid chamber so that its second end faces downwards;

(d) inserting the liquid chamber into the electrolytic chamber until the base comes into direct or indirect contact with the open end of the electrolytic chamber; and

(e) removably fastening the base to the electrolytic chamber thereby sealing the electrolyte in the electrolytic chamber.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### THE PREFERRED EMBODIMENT

A preferred embodiment of the invention will now be described, by way of example only, with reference to the accompanying drawings in which:

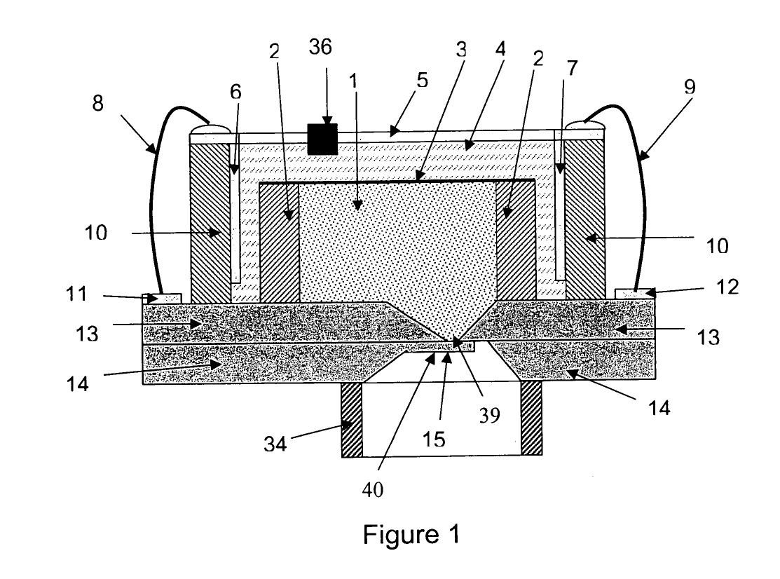

FIG. 1 is a schematic cross-sectional view of a first preferred embodiment of the liquid delivering device of the present invention, showing the one-way valve in its closed position;

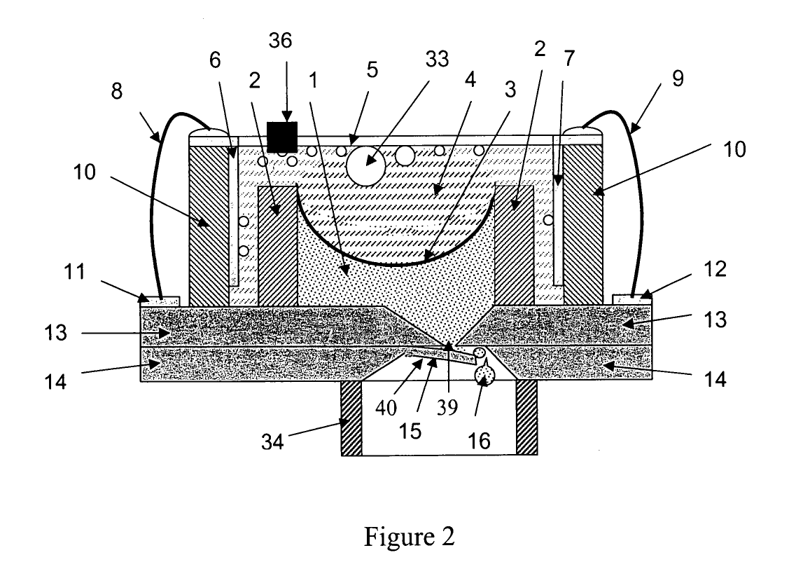

FIG. 2 is a schematic cross-sectional view of the device of FIG. 1, showing the one-way valve in its open position;

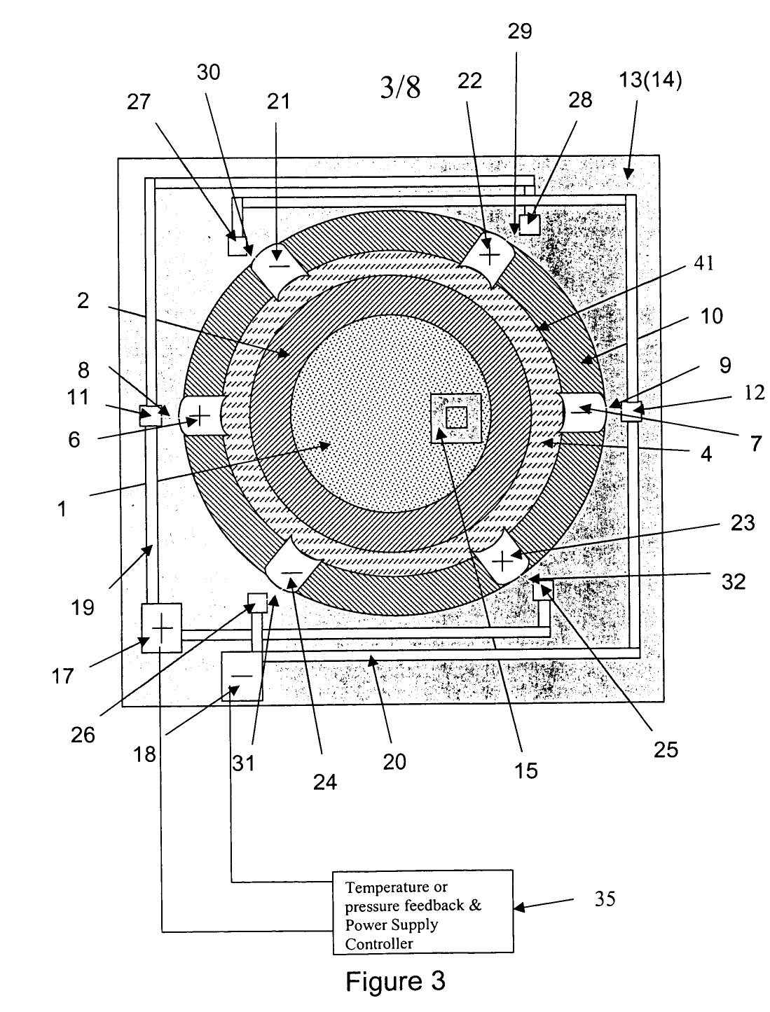

FIG. 3 is a schematic plan view of the device of FIG. 1;

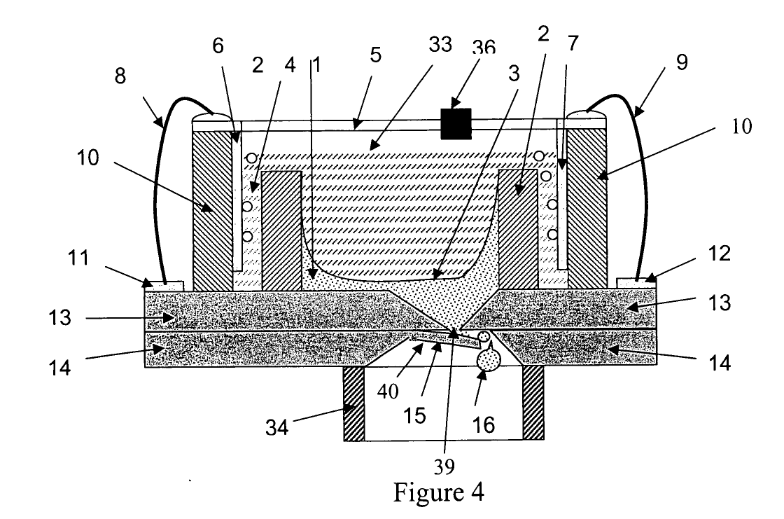

FIG. 4 is a schematic cross-sectional view of the device of FIG. 1 showing the deformable membrane 3 having almost completely squeezed the liquid 1 from the liquid chamber 2;

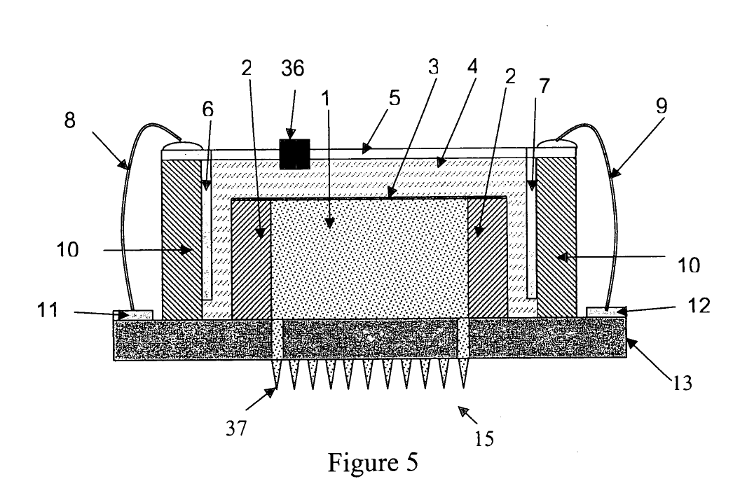

FIG. 5 is a schematic cross-sectional view of a second preferred embodiment of the liquid delivering device of the present invention;

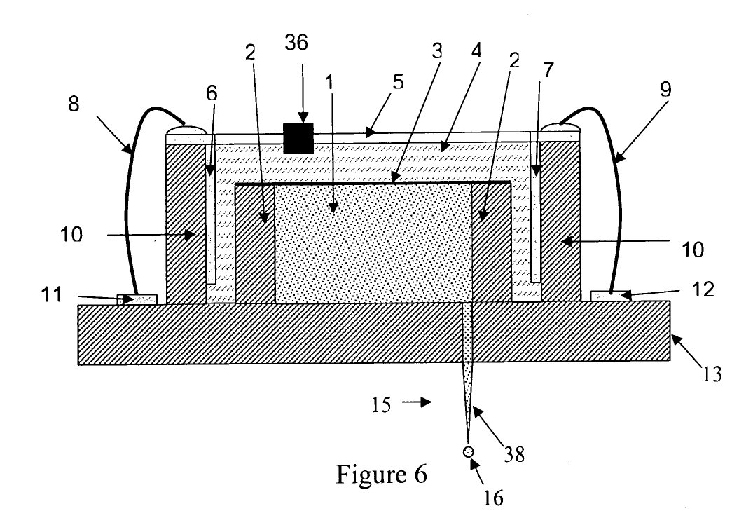

FIG. 6 is a schematic cross-sectional view of a third preferred embodiment of the liquid delivering device of the present invention;

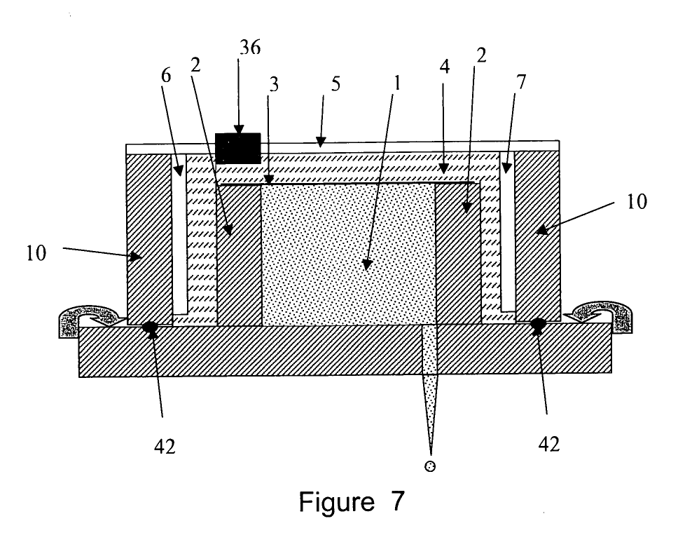

FIG. 7 is a schematic cross-sectional view of a fourth preferred embodiment of the liquid delivering device of the present invention; and

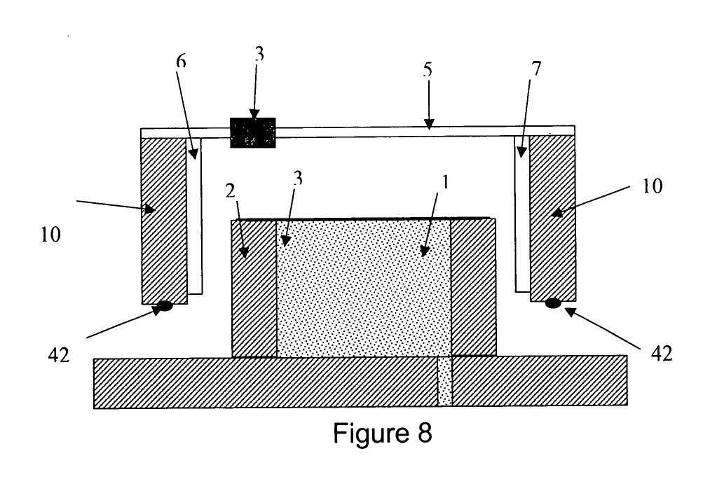

FIG. 8 is a schematic cross-sectional view of the device of FIG. 7 showing the base and liquid chamber separated from the electrolytic chamber.

Referring to the drawings, FIG. 1 shows a schematic cross-sectional view of a first preferred embodiment of the liquid delivering device of the present invention. That device comprises a sealed electrolytic chamber 10 which is adapted to contain an electrolyte 4 and which has three pairs of electrodes 6,7 at least partially in contact with the electrolyte. The device also has a liquid chamber 2 which is adapted to contain a liquid 1, pressure transfer means 3 separating the electrolytic chamber 10 from the liquid chamber 2, and administering means 15 adapted to release the liquid 1 from the liquid chamber under the influence of pressure applied to the liquid chamber.

As shown in FIG. 2, when a current is passed through the electrolyte 4 via the electrodes 6, 7 a number of events take place. Firstly, gas 33 is generated from the electrolyte 4 thereby increasing pressure inside the electrolytic chamber 10. Next, that increase in pressure is transferred to the liquid chamber 2 via the pressure transfer means 3. Finally, a determinable amount of the liquid 1 is released from the liquid chamber 2 via the administering means 15. In this preferred embodiment the liquid chamber 2 is contained wholly within the electrolytic chamber 10, both chambers are coaxial, are substantially cylindrical in shape, and share a common base 13. The chambers are made at least partially from substantially rigid materials such as metals, alloys, polymeric or ceramic materials. In this embodiment, the chambers are made from glass cylinders bonded anodically to the base 13. The top of the electrolytic chamber is sealed with a cover 5 made from a substantially rigid material, such as metals, alloys, polymeric or ceramic materials.

In this preferred embodiment, the device is constructed using microfabrication techniques, injection moulding and/or mechanical machining.

The pressure transfer means 3 is, in this preferred embodiment, a deformable membrane. However, alternative embodiments may employ any type of pressure transfer means including a bellows, a piston, a diaphragm, a bladder or the like.

The deformable membrane of the preferred embodiment is at least partially made from hyper-elastic materials, materials with high elasticity and/or materials with high plasticity. In addition, the membrane 3 is at least partially made from one or more materials which resist permeation by gas, liquid, semi-solids and/or gels.

As shown in FIG. 1, in this embodiment, the membrane 3 is circular in shape and is fixed at its perimeter to an upper edge of the liquid chamber 2. As shown in FIG. 2, when pressure is exerted from gas generated in the electrolytic chamber 10, the central part of the membrane deforms into the liquid chamber 2. As shown in FIG. 4, because the membrane is made from highly elastic materials, in its fully deformed state it can extend right to the base of the liquid chamber, squeezing all of the liquid out.

As shown in FIG. 1, the administering means 15 of the preferred embodiment is adapted to release the liquid 1 from the liquid chamber 2 and administer it to a subject either directly or indirectly. In the first preferred embodiment the administering means comprises a one-way check valve 15 made from a first wafer 13 having an opening 39 and a second wafer 14 having a deformable flange 40. In this embodiment, the first and second wafers 13, 14 are monolithic silicon wafers manufactured using micromachining techniques. Other forms of one-way valve are also envisaged. The one-way check valve is a closed check valve and the deformable flange 15 is biased towards a closed position in which it covers the opening 39. As shown in FIG. 2, when the liquid 1 in the liquid chamber 2 reaches a threshold pressure, the deformable flange 40 deforms outwardly, uncovering the opening 39 and releasing liquid from the liquid chamber.

As shown in FIGS. 1, 2 and 4, the one-way check valve is in communication with a connector 34. In this embodiment, the connector is a cylindrical glass tube centred on the valve 15. The inner diameter of the tube 34 is slightly larger than the size of the valve 15 and is used to connect a polymeric tube for transmitting the liquid 1 to the subject. When the liquid is a drug liquid, the polymeric tube transmits the drug liquid into an injection location on the subject.

Turning now to FIG. 5, in the second preferred embodiment of the liquid delivering device of the present invention, the administering means 15 takes the form of a plurality of micro-injection needles 37. In this embodiment, the needles are attached to or fabricated at the substrate silicon piece 13 and allow the device to be attached to or implanted inside the subject, delivering the liquid, such as a drug liquid, directly.

Turning to FIG. 6, in the third preferred embodiment of the liquid delivering device of the present invention, the administering means 15 takes the form of a nozzle hole 38. In addition, the liquid chamber 2, electrolytic chamber 5 and base 13 are formed as a single unit. This unified embodiment may be made by injection moulding polymeric materials, ceramic materials, metallic materials or other material suitable for injection moulding. It may also be made using conventional mechanical machining techniques.

It can be seen from the plan view of FIG. 3 that the three pairs of electrodes 6,7,21,22,23,24 are spaced at regular intervals within the electrolytic chamber 10. The anodes and cathodes are arranged alternatively and are connected to the chamber's internal wall 41. The even distribution of the anodes and cathodes ensures that they are in contact with the electrolyte 4 no matter what orientation the device is held in. Although this embodiment includes three pairs of electrodes, fewer, or greater numbers of electrodes are also envisaged. The electrodes may be made from metals, metal alloys, conductive metal oxides, conductive metal halides, conductive silicides, conductive borides, conductive carbides, conductive nitrides, multi-layer conductors or any other conductive material.

As shown in FIGS. 1 and 3, electrode 6 is wire-bonded to pad 11 via wire 8. Electrode 7 is wire-bonded to pad 12 via wire 9. As shown in FIG. 3 electrodes 21, 22, 23 and 24 are wire-bonded to pads 25, 26, 27 and 28 in a similar way through wires 29, 30, 31 and 32. In turn, pads 28, 11 and 25 are connected to pad 17 via a lead 19. Similarly, pads 27, 12 and 26 are connected to pad 18 via another lead 20.

Pads 17 and 18 are connected to a power supply 35. In the first, and possibly the second preferred embodiments, the pads and leads are fabricated in the surface of the base 13 using micromachining technology prior to the assembly of the device.

As shown in FIG. 1, the preferred embodiment includes a sensor 36 connected to the cover 5 of the electrolytic chamber 10. That sensor is adapted to sense internal pressure and/or internal temperature of the electrolytic chamber. The sensor is in communication with the power supply 35 shown in FIG. 3 and provides feedback to a current controller within that power source regarding the internal pressure and temperature of the electrolytic chamber. By adjusting the current supplied to the electrolyte the device is able to deliver a precise volume of liquid at a precise flow rate. This is because the volume and pressure of the gas produced by the electrolysis may be accurately calculated using Faraday's law. As such, the pressure of the gas and its rate of change may be controlled precisely by adjusting the current flowing through the electrolyte 4 to deliver a precise volume of liquid at a precise flow rate.

The inventors have used the device to administer liquids at rates as low as 3.2 nano litres per second. However, with minor variations to the device, smaller or greater flow rates may be achieved.

In one embodiment, at least part of the device would be adapted to be used only once and then disposed. For example, the liquid chamber 2 may be designed to be filled with liquid only once and the electrolytic chamber 10 may be adapted to be filled with electrolyte only once. One example of a partially disposable form of the present invention is shown in FIGS. 7 and 8.

Referring firstly to FIG. 7, in this embodiment the electrolytic chamber 10 is removably connected to the base 13 by means of a fastening mechanism (not shown). An appropriate seal 42 such as a rubber o-ring is placed between the electrolytic chamber and the base 13 to ensure that the electrolyte 4 does not escape from the chamber. As shown in FIG. 8, once the device has been used, the fastening mechanism can be released and the electrolytic chamber may be separated from the combined base 13 and liquid chamber 2. In this embodiment the used liquid chamber may then be disposed and replaced with a new liquid chamber.

To re-assemble the device with the new liquid chamber 2, the electrolytic chamber is inverted and filled with electrolyte 4. The new liquid chamber 2 and base 13 are also inverted and inserted into the electrolytic chamber 10 until the base 13 comes into contact with the electrolytic chamber 10. The seals 42 between the electrolytic chamber and the base 13 ensure that the electrolyte does not escape the electrolytic chamber. The base and the electrolytic chamber are then fastened together using the fastening mechanism.

In this way the electrolytic chamber portion of the device may be re-used, limiting the disposable part of the device to the liquid chamber and base, thereby reducing its operation cost.

Any of the embodiments of this device may form just one component of a complete liquid delivery system. The device may therefore be detached from the rest of the system and disposed, or reused after filling with a new liquid. When the liquid is a drug liquid, the present invention is applicable in a variety of medical and bio-medical fields. Use of the device would be to administer insulin to diabetics. It is not expensive to produce because the knowledge used to fabricate it is compatible with those used in the semi-conductor industry or precision engineering.

In its preferred form, the gas liberated from the electrolyte is non-toxic.

All four embodiments of the present invention may be used to administer liquid to a subject using a number of steps. The first step involves providing the liquid delivering device as described above. The next step involves connecting the administering means to the subject. In the case of the second preferred embodiment shown in FIG. 5 where the administering means comprises the plurality of micro-injection needles 37, this step involves inserting at least part of the plurality of needles directly into the subject. In the case of the first, third and fourth embodiments shown in FIGS. 2, 6 and 7, the step of connecting the administering means to the subject would involve connecting one end of connecting tube 34 to the one-way valve or nozzle and connecting the other end of the tube 34 to the subject via an appropriate injecting device.

The first three embodiments of the present invention may be manufactured according to a method comprising a number of steps. The first step involves providing a substantially planar base having an outlet in which the outlet is adapted to allow selective passage of a liquid through it. The next step involves providing a liquid chamber made up of a substantially tubular housing which has a first open end and a second open end. The next step involves bonding the first open end of the liquid chamber housing to the base over the outlet. The next step involves providing an electrolytic chamber comprising a substantially tubular housing of a greater diameter and length than the liquid chamber housing and having a first open end and a second open end, and at least one pair of electrodes extending at least partially from an inner wall of the housing. The next step involves bonding the first open end of the electrolytic chamber housing to the base over the liquid chamber so as to house the liquid chamber. The next step involves providing a substantially planar deformable membrane which has an inner section and an outer section. The next step involves bonding the outer section of the membrane to the second end of the liquid chamber housing. The next step involves providing a substantially planar cover having an inner section and an outer section. The next step involves bonding the outer section of the cover to the second end of the liquid chamber housing.

Once the above steps have been done, the device is ready to be primed. The base is preferably made with a one-way liquid inserting valve which allows one-way access of a liquid into the inside of the liquid chamber. The method then involves filling the liquid chamber with the liquid, such as a drug liquid, via that valve.

Alternatively, the base may be made with a liquid inserting hole in it. Once the liquid chamber has been filled with the liquid via that hole, it can then be sealed.

In a similar way the electrolytic chamber may be (at least partially) filled with a electrolyte via a hole or one way valve in the cover. The preferred manufacturing method uses a resealable hole for the electrolytic chamber and a one-way valve for the liquid chamber.

In an alternative method, the step of providing an electrolytic chamber comprising a substantially tubular housing of a greater diameter and length than the liquid chamber housing and having a first open end and a second open end, and at least one pair of electrodes extending at least partially from an inner wall of the housing is replaced by the step of providing an electrolytic chamber comprising a substantially tubular housing of a greater diameter and length than the liquid chamber housing and having a first open end and a second open end. In turn, the step of providing a substantially planar cover having an inner section and an outer section is replaced by the step of providing a substantially planar cover having an inner section and an outer section and at least one pair of electrodes extending at least partially therefrom.

One particular application of this method of manufacture may be described as follows:

A silicon substrate base is formed with a one-way inlet valve and a one-way outlet valve (or other administering means) in it, using microfabrication technique. An electrolytic chamber is formed from a Pyrex glass hollow cylinder. A set of three pairs of electrodes are made in the internal wall of the Pyrex cylinder. One end of the cylinder is bonded anodically to the base. The liquid chamber housing is made from another Pyrex glass hollow cylinder having a smaller diameter and height than the electrolytic chamber housing. The liquid chamber housing is inserted into the electrolytic chamber housing and is bonded anodically to the silicon wafer base. The one-way outlet valve (or other administering means) in the silicon base is located within the bonded area of the liquid chamber Pyrex glass hollow cylinder. The other end of the liquid chamber glass cylinder is sealed with a non-permeable flexible or elastic membrane. The membrane is fixed only at its perimeter to the top end wall of the liquid chamber cylinder. Its central part may be deformed downwards into the liquid chamber by pressure exerted from above.

The other end of the electrolytic chamber glass cylinder is then sealed by a plate made of some rigid material, thereby sealing the electrolyte inside. The plate may be made from any appropriate metal, alloy, polymeric or ceramic material and has an electrolyte inserting hole in it. The electrodes disposed at the inner wall of the electrolytic chamber glass cylinder are then connected through wire bonding to a set of pads which had been previously fabricated at the silicon substrate piece.

The liquid chamber is then filled with liquid (such as a drug liquid) via the one-way inlet valve. The electrolytic chamber is then (at least partially) filled with electrolyte via the electrolyte inserting hole. The hole is then sealed, thereby sealing the electrolyte in the electrolytic chamber.

As described above, the fourth embodiment of the present invention is directed at a form in which the liquid chamber and base are removably attachable to the electrolytic chamber for the purpose of enabling the two parts to be separately disposable. This fourth embodiment may be manufactured according to a preferred method. That method involves a number of steps. The first step involves providing a substantially planar base which has an outlet, the outlet being adapted to allow selective passage of a liquid therethrough. The next step involves providing a liquid chamber comprising a substantially tubular housing which has a first open end and a second open end. The next step involves bonding the first open end of the liquid chamber housing to the base over the outlet. The next step involves providing a substantially planar deformable membrane which has an inner section and an outer section. The next step involves bonding the outer section of the membrane to the second end of the liquid chamber housing thereby sealing the liquid in the liquid chamber. The next step involves filling the liquid chamber with a liquid. The next step involves providing an electrolytic chamber having:

(i) a substantially tubular housing of a greater diameter and length than the liquid chamber housing and having an open end and a sealed end; and

(ii) at least one pair of electrodes extending at least partially from an inner wall of the housing.

The next step involves inverting the electrolytic chamber housing so that its open end faces upwards. The next step involves at least partially filling the electrolytic chamber housing with an electrolyte. The next step involves inverting the liquid chamber so that its second end faces downwards. The next step involves inserting the liquid chamber into the electrolytic chamber until the base comes into direct or indirect contact with the open end of the electrolytic chamber. The final step involves removably fastening the base to the electrolytic chamber using the fastening device, thereby sealing the electrolyte in the electrolytic chamber.

Alternatively, when the fourth embodiment of the present invention takes the form of:

(a) a substantially planar base having an outlet therein, the outlet adapted to allow selective passage of a liquid therethrough;

(b) a liquid chamber comprising a substantially tubular housing having a first end sealed by a substantially planar deformable membrane, a second end bonded to the base over the outlet, and being filled with a liquid; and

(c) a separate electrolytic chamber comprising:
(i) a substantially tubular housing of a greater diameter and length than the liquid chamber housing and having an open end and a sealed end; and
(ii) at least one pair of electrodes extending at least partially from an inner wall of the housing,
   it may be assembled according to a method comprising another set of steps. The first step involves inverting the electrolytic chamber housing so that its open end faces upwards. The next step involves at least partially filling the electrolytic chamber housing with an electrolyte. The next step involves inverting the liquid chamber so that its second end faces downwards. The next step involves inserting the liquid chamber into the electrolytic chamber until the base comes into direct or indirect contact with the open end of the electrolytic chamber. The final step involves removably fastening the base to the electrolytic chamber thereby sealing the electrolyte in the electrolytic chamber.

It will be appreciated from the foregoing discussion that the present invention has a number of advantages.

First, it is able to be made in a compact size. This is because by housing the liquid chamber at least partially within the electrolytic chamber device maximises its use of space.

Second, because of the design of the liquid chamber and the selection of the deformable membrane made from materials which are highly elastic, the membrane may be deformed under pressure from the electrolytic chamber to such an extent that it extends to the bottom of the liquid chamber housing, completely squeezing out the liquid.

Third, at least part of the device can be disposable. Because the device can be made in large volumes for minimal cost using microfabrication techniques, the device can be made inexpensively enough to be used only once. This helps to maintain the sterility of the administering means.

Fourth, the delivered liquid is free from gas bubbles. This is particularly important for medical applications. This advantage is achieved by separating the electrolysis and liquid chambers by the membrane. In this way, the gas which accumulates in the electrolytic chamber cannot penetrate the membrane into the liquid chamber. The membrane does, however allow the pressure of the electrolytic chamber to be transmitted through to the liquid chamber. In addition, in the preferred embodiment the drug liquid in the liquid chamber is fully degased both before and after the liquid chamber is filled.

Fifth, the device does not have the backflow problems of the prior art. Because of the one-way valve or similar administering means, the backflow of drug liquid or other contaminant does not occur. The one-way valve also ensures that the pressure of the liquid in the liquid chamber is maintained even when the device is turned off so that contaminants do not enter the liquid chamber.

Sixth, the device is capable of dosing an exact amount of drug liquid at a precise flow rate. Because the volume and pressure of the gas generated in the electrolyte can be calculated exactly by referring to the current passing through it and Faraday's law, the amount of liquid being released from the device can be monitored and controlled very precisely.

Seventh, the device is orientation independent. Because the electrodes are distributed evenly throughout the inner wall of the electrolytic chamber housing, they will maintain contact with the electrolyte no matter what orientation the device is positioned in.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A liquid delivering device comprising:
(a) a sealed electrolytic chamber adapted to contain an electrolyte and having at least one pair of electrodes at least partially inside said chamber;
(b) a liquid chamber housed at least partially within said electrolytic chamber and adapted to contain a liquid, said liquid chamber comprising a base and a sidewall each formed of a substantially rigid material;
(c) pressure transfer means separating said electrolytic chamber from said liquid chamber; and
(d) administering means adapted to release said liquid from said liquid chamber under influence of pressure applied to said liquid chamber
whereby when the electrolytic chamber contains an electrolyte, the liquid chamber contains a liquid and a current is passed through said electrolyte via said electrodes:
(i) gas is generated from the electrolyte thereby increasing pressure inside the electrolytic chamber;
(ii) that increase in pressure is transferred to said liquid chamber via said pressure transfer means; and
(iii) a determinable amount of said liquid is released from said liquid chamber via said administering means.

2. A device according to claim 1 wherein said pressure transfer means comprises one or more of:
(a) a deformable membrane;
(b) a bellow;
(c) a piston;
(d) a diaphragm; and
(e) a bladder.

3. A device according to claim 2 wherein said pressure transfer means is a deformable membrane and said membrane is at least partially made from one or more of:
(a) hyper-elastic materials;
(b) materials with high elasticity; and
(c) materials with high plasticity.

4. A device according to claim 2 or claim 3 wherein said pressure transfer means is a deformable membrane and said membrane is at least partially made from one or more materials which resist permeation by one or more of:
(a) gas;
(b) liquid;
(c) semi solids; and
(d) gels.

5. A device according to any one of claims 2 to 4 wherein said pressure transfer means is a deformable membrane and said membrane is at least partially joined to an upper edge of said liquid chamber.

6. A device according to any one of the preceding claims wherein said device is adapted to release said liquid at a flow rate of between 1 and 1000 micro litres per second.

7. A device according to any one of the preceding claims wherein said electrolytic and liquid chambers are substantially co-axial.

8. A device according to claim 7 wherein the sidewall of the liquid chamber is a substantially cylindrical wall.

9. A device according to claim 8 wherein one or both of said electrolytic and liquid chambers are substantially cylindrical in shape.

10. A device according to claim 8 or claim 9 wherein said liquid chamber is contained wholly within said electrolytic chamber.

11. A device according to claim 10 wherein said electrolytic and liquid chambers share a common base.

12. A device according to any one of the preceding claims wherein said administering means comprises one or more of:
(a) one or more one-way valves;
(b) one or more needles; and
(c) one or more nozzle holes.

13. A device according to claim 12 wherein said one or more needles comprises one or more micro injection needles.

14. A device according to claim 13 wherein said administering means comprises a one-way valve and said one-way valve comprises a one-way check valve.

15. A device according to claim 14 wherein said one-way check valve comprises:
(a) a first wafer having an opening; and
(b) a second wafer having a deformable flange;
wherein said first and second wafers are at least partially bonded to one another so that said deformable flange covers said opening.

16. A device according to claim 15 wherein said deformable flange is biased towards a closed position in which it covers said opening.

17. A device according to claim 16 wherein when said liquid in said liquid chamber reaches a threshold pressure said deformable flange deforms outwardly, uncovering said opening and releasing a determinable amount of said liquid from said liquid chamber.

18. A device according to any one of claims 15 to 17 wherein one or both of said first and second wafers are silicon wafers.

19. A device according to claim 18 wherein one or both of said first and second wafers are monolithic silicon wafers.

20. A device according to claim 19 wherein one or both of said first and second wafers are manufactured using micromachining techniques.

21. A device according to any one of the preceding claims wherein said administering means is formed in a base of said liquid chamber.

22. A device according to any one of the preceding claims wherein said electrolytic chamber further includes a sensor adapted to sense one or more of:
(a) internal pressure; and
(b) internal temperature of said electrolytic chamber.

23. A device according to claim 22 wherein said sensor is in communication with a current controller.

24. A device according to claim 23 wherein said sensor is adapted to provide information to said current controller regarding one or more of the:
(a) internal pressure; and
(b) internal temperature of said electrolytic chamber.

25. A device according to any one of the preceding claims wherein said device is adapted to release said liquid at a flow rate of between 1 and 1000 nano litres per second.

26. A device according to any one of claims 1 to 24 wherein said device is adapted to release said liquid at a flow rate of between 1 and 1000 pico litres per second.

27. A device according to any one of the preceding claims wherein at least part of said device is adapted to be used only once.

28. A device according to claim 27 wherein said liquid chamber is adapted to be filled with said liquid only once.

29. A device according to claim 27 or claim 28 wherein said electrolytic chamber is adapted to be filled with said electrolyte only once.

30. A device according to any one of the preceding claims wherein said electrolytic chamber comprises:
(a) an electrolytic chamber housing defining an electrolytic chamber cavity; and
(b) at least one pair of electrodes extending at least partially into said electrolytic chamber cavity.

31. A device according to claim 30 wherein said at least one pair of electrodes are spaced at regular intervals within said electrolytic chamber cavity.

32. A device according to claim 31 wherein said at least one pair of electrodes comprises two pairs of electrodes spaced at regular intervals within said electrolytic chamber cavity.

33. A device according to claim 31 wherein said at least one pair of electrodes comprises three pairs of electrodes spaced at regular intervals within said electrolytic chamber cavity.

34. A device according to claim 31 wherein said at least one pair of electrodes comprises four pairs of electrodes spaced at regular intervals within said electrolytic chamber cavity.

35. A device according to any one of claims 30 to 34 wherein said electrodes are at least partially made from one or more of:
(a) metals;
(b) metal alloys;
(c) conductive metal oxides;
(d) conductive metal halides;
(e) conductive silicides;
(f) conductive borides;
(g) conductive carbides;
(h) conductive nitrides;
(i) multi-layer conductors; and
(j) other conductive materials.

36. A device according to any one of claims 30 to 35 wherein said electrolytic chamber housing is at least partially formed of a substantially rigid material.

37. A device according to any one of the preceding claims wherein the sidewall of said liquid chamber is a substantially cylindrical sidewall defining a liquid chamber cavity.

38. A device according to any one of the preceding claims wherein said substantially rigid material is manufactured using one or more of the following techniques:
(a) microfabrication techniques;
(b) injection moulding; and
(c) mechanical machining.

39. A device according to any one of the preceding claims wherein said substantially rigid material is at least partially made from one or more of:
(a) polymeric materials;
(b) ceramic materials;
(c) metals;
(d) metal alloys; and
(e) glass.

40. A device according to any one of the preceding claims wherein said liquid comprises a drug dispersed or dissolved in a liquid.

41. A device according to claim 40 wherein said drug is insulin.

42. A device according to any one of the preceding claims wherein said gas liberated from said electrolyte is non-toxic.

43. A method of administering a liquid to a subject, said method comprising the steps of:
(a) providing a liquid delivering device comprising:
(i) a sealed electrolytic chamber containing an electrolyte and having at least one pair of electrodes at least partially in contact with said electrolyte;
(ii) a liquid chamber housed at least partially within said electrolytic chamber and containing a liquid, said liquid chamber comprising a base and a sidewall each formed of a substantially rigid material;
(iii) pressure transfer means separating said electrolytic chamber from said liquid chamber; and
(iv) administering means adapted to release said liquid from said liquid chamber under influence of pressure applied to said liquid chamber
(b) connecting said administering means to said subject;
(c) passing a current through said electrolyte via said electrodes causing:
(i) gas to be generated from said electrolyte thereby increasing pressure inside the electrolytic chamber;
(ii) that increase in pressure to be transferred to said liquid chamber via said pressure transfer means; and
(iii) a determinable amount of said liquid to be released from said liquid chamber via said administering means.

44. A method according to claim 43 wherein said administering means comprises one or more injection needles and the step of connecting said administering means to said subject comprises inserting at least part of said one or more needles directly into said subject.

45. A method according to claim 43 wherein said administering means comprises one or more of:
(a) a one way valve; and
(b) a nozzle
and wherein said step of connecting said administering means to said subject comprises:
(a) connecting a first end of a connecting tube to said one way valve or nozzle; and
(b) connecting a second end of said connecting tube to said subject.

46. A method of manufacturing a liquid delivering device, said method comprising the steps of:
(a) providing a substantially planar base having an outlet therein, said outlet adapted to allow selective passage of a liquid therethrough;
(b) providing a liquid chamber comprising a substantially rigid and substantially tubular housing having a first open end and a second open end;
(c) bonding said first open end of said liquid chamber housing to said base over said outlet;
(d) providing an electrolytic chamber comprising:
(i) a substantially tubular housing of a greater diameter and length than said liquid chamber housing and having a first open end and a second open end; and
(ii) at least one pair of electrodes at least partially inside said housing;
(e) bonding said first open end of said electrolytic chamber housing to said base over said liquid chamber so as to house said liquid chamber;
(f) providing a substantially planar deformable membrane having an inner section and an outer section;
(g) bonding said outer section of said membrane to said second end of said liquid chamber housing;
(h) providing a substantially planar cover having an inner section and an outer section; and
(i) bonding said outer section of said cover to said second end of said liquid chamber housing.

47. A method according to claim 46 including the additional steps of:
(j) filling said liquid chamber with a liquid; and
(k) at least partially filling said electrolytic chamber housing with an electrolyte.

48. A method according to claim 46 or claim 47 wherein said outlet comprises one or more of:
(a) one or more one-way valves;
(b) one or more needles; and
(c) one or more nozzle holes.

49. A method according to claim 48 wherein said one or more needles comprises one or more micro injection needles.

50. A method according to claim 48 wherein said administering means comprises a one-way valve and said one-way valve comprises a one-way check valve.

51. A method according to claim 50 wherein said one-way check valve comprises:
(a) a first wafer having an opening; and
(b) a second wafer having a deformable flange
wherein said first and second wafers are at least partially bonded to one another so that said deformable flange covers said opening.

52. A method according to claim 51 wherein said deformable flange is biased towards a closed position in which it covers said opening.

53. A method according to claim 52 wherein when said liquid in said liquid chamber reaches a threshold pressure said deformable flange deforms outwardly, uncovering said opening and releasing a determinable amount of said liquid from said liquid chamber.

54. A method according to any one of claims 51 to 53 wherein one or both of said first and second wafers are silicon wafers.

55. A method according to claim 54 wherein one or both of said first and second wafers are monolithic silicon wafers.

56. A method according to claim 55 wherein one or both of said first and second wafers are manufactured using micromachining techniques.

57. A method according to any one of claims 51 to 56 wherein said wafers constitute said base.

58. A method according to any one of claims 46 to 57 wherein said electrolytic and liquid chambers are substantially co-axial.

59. A method according to claim 58 wherein one or both of said electrolytic and liquid chambers are substantially cylindrical in shape.

60. A method according to claim 59 wherein said liquid chamber is contained wholly within said electrolytic chamber.

61. A method according to any one of claims 46 to 60 wherein said electrolytic chamber housing is at least partially formed of a substantially rigid material.

62. A method according to any one of claims 46 to 61 wherein said substantially rigid material is manufactured using one or more of the following techniques:
(a) microfabrication techniques;
(b) injection moulding; and
(c) mechanical machining.

63. A method according to any one of claims 1 to 61 wherein said substantially rigid material includes glass.

64. A method according to any one of claims 1 to 61 wherein said substantially rigid material includes:
(a) polymeric materials;
(b) ceramic materials; and
(c) a metal or an alloy.

65. A method according to any one of claims 43 to 64 wherein said liquid comprises a drug dispersed or dissolved in a liquid.

66. A method according to claim 65 wherein said drug liquid is insulin.

67. A method according to any one of claims 46 to 66 wherein said deformable membrane is at least partially made from one or more of:
(a) hyper-elastic materials;
(b) materials with high elasticity; and
(c) materials with high plasticity.

68. A method according to any one of claims 46 to 67 wherein said deformable membrane is at least partially made from one or more materials which resist permeation by one or more of:
(a) gas;
(b) liquid;
(c) semi solids; and
(d) gels.

69. A method according to any one of claims 46 to 68 wherein said cover further includes a sensor adapted to sense one or more of the:
(a) internal pressure; and
(b) internal temperature of said electrolytic chamber.

70. A method according to claim 69 wherein said sensor is in communication with a current controller.

71. A method according to claim 70 wherein said sensor is adapted to provide information to said current controller regarding one or more of the:
(a) internal pressure; and
(b) internal temperature of said electrolytic chamber.

72. A method according to any one of claims 46 to 71 wherein said step of:
(c) bonding said first open end of said liquid chamber housing to said base over said outlet
comprises anodically bonding said liquid chamber housing to said base.

73. A method according to any one of claims 46 to 72 wherein said step of:
(e) bonding said first open end of said electrolytic chamber housing to said base over said liquid chamber so as to house said liquid chamber
comprises anodically bonding said electrolytic chamber housing to said base.

74. A method according to any one of claims 46 to 73 wherein said step of:
(j) filling said liquid chamber with a liquid includes the preliminary or subsequent additional step of de-gassing said liquid.

75. A method according to any one of claims 46 to 74 wherein said at least one pair of electrodes are spaced at regular intervals around said inner wall of said electrolytic chamber housing.

76. A method according to claim 75 wherein said at least one pair of electrodes comprises two pairs of electrodes spaced at regular intervals around said inner wall of said electrolytic chamber housing.

77. A method according to claim 75 wherein said at least one pair of electrodes comprises three pairs of electrodes spaced at regular intervals around said inner wall of said electrolytic chamber housing.

78. A method according to claim 75 wherein said at least one pair of electrodes comprises four pairs of electrodes spaced at regular intervals around said inner wall of said electrolytic chamber housing.

79. A method according to any one of claims 67 to 78 wherein said electrodes are at least partially made from one or more of:
(a) metals;
(b) conductive metal oxides;
(c) conductive metal halides;
(d) conductive silicides;
(e) conductive borides;
(f) conductive carbides;
(g) conductive nitrides;
(h) multilayer conductors; and
(i) other conductive materials.

80. A method according to any one of claims 67 to 79 wherein said device is adapted to release said liquid at a flow rate of between 1 and 1000 micro litres per second.

81. A method according to any one of claims 67 to 79 wherein said device is adapted to release said liquid at a flow rate of between 1 and 1000 nano litres per second.

82. A method according to any one of claims 67 to 79 wherein said device is adapted to release said liquid at a flow rate of between 1 and 1000 pico litres per second.

83. A method according to any one of claims 67 to 82 wherein said liquid chamber is adapted to be filled with said liquid only once.

84. A method according to any one of claims 67 to 83 wherein said electrolytic chamber is adapted to be filled with said electrolyte only once.

85. A device according to claim 27 wherein said electrolytic chamber is adapted to be removably attachable to said base.

86. A device according to claim 85 further including a sealing component located between said electrolytic chamber and said base when they are attached.

87. A device according to claim 85 or claim 86 further including a fastening mechanism adapted to removably attach said electrolytic chamber to said base.

88. A method of manufacturing a liquid delivering device, said method comprising the steps of:
(a) providing a substantially planar base having an outlet therein, said outlet adapted to allow selective passage of a liquid therethrough;
(b) providing a liquid chamber comprising a substantially rigid and substantially tubular housing having a first open end and a second open end;
(c) bonding said first open end of said liquid chamber housing to said base over said outlet;
(d) providing a substantially planar deformable membrane having an inner section and an outer section;
(e) bonding said outer section of said membrane to said second end of said liquid chamber housing;
(f) providing an electrolytic chamber comprising:
(i) a substantially tubular housing of a greater diameter and length than said liquid chamber housing and having an open end and a sealed end; and
(ii) at least one pair of electrodes at least partially inside said housing;
(g) inverting said electrolytic chamber housing so that its open end faces upwards;
(h) at least partially filling said electrolytic chamber housing with an electrolyte;
(i) inverting said liquid chamber so that its second end faces downwards;
(j) inserting said liquid chamber into said electrolytic chamber until said base comes into direct or indirect contact with said open end of said electrolytic chamber; and
(k) removably fastening said base to said electrolytic chamber using said fastening device, thereby sealing said electrolyte in said electrolytic chamber.

89. A method of assembling a liquid delivering device comprising:
(a) a substantially planar base having an outlet therein, said outlet adapted to allow selective passage of a liquid therethrough;
(b) a liquid chamber comprising a substantially rigid and substantially tubular housing having a first end sealed by a substantially planar deformable membrane, a second end bonded to said base over said outlet, and being filled with a liquid; and
(c) a separate electrolytic chamber comprising:
(i) a substantially tubular housing of a greater diameter and length than said liquid chamber housing and having an open end and a sealed end; and
(ii) at least one pair of electrodes at least partially inside said housing;
said method comprising the steps of:
(a) inverting said electrolytic chamber housing so that its open end faces upwards;
(b) at least partially filling said electrolytic chamber housing with an electrolyte;
(c) inverting said liquid chamber so that its second end faces downwards;
(d) inserting said liquid chamber into said electrolytic chamber until said base comes into direct or indirect contact with said open end of said electrolytic chamber; and
(e) removably fastening said base to said electrolytic chamber thereby sealing said electrolyte in said electrolytic chamber.

90. A method according to claim 46 wherein step (f) is replaced by the step of:
(f1) providing an electrolytic chamber comprising a substantially tubular housing of a greater diameter and length than said liquid chamber housing and having a first open end and a second open end;
and step (h) is replaced by the step of
(h1) providing a substantially planar cover having an inner section and an outer section and at least one pair of electrodes extending at least partially therefrom.

91. A method according to claim 47 wherein said step of (j) filling said liquid chamber with a liquid comprises the steps of:
(a) providing a one-way liquid inserting valve in said base, said valve adapted to provide one way liquid access to the inside of the liquid chamber; and
(b) filling said liquid chamber with a liquid via said valve.

92. A method according to claim 47 wherein said step of (j) filling said liquid chamber with a liquid comprises the steps of:
(a) providing a liquid inserting hole in said base;
(b) filling said liquid chamber with a liquid via said hole; and
(c) sealing said hole thereby sealing said liquid in said liquid chamber.

93. A method according to any one of claims 47, 91 or 92 wherein said step of (k) at least partially filling said electrolytic chamber housing with an electrolyte comprises the steps of:
(a) providing an electrolyte inserting hole in said cover;
(b) at least partially filling said electrolytic chamber housing with an electrolyte via said hole; and
(c) sealing said hole thereby sealing said electrolyte in said electrolytic chamber.

94. A method according to any one of claims 47, 91 or 92 wherein said step of (k) at least partially filling said electrolytic chamber housing with an electrolyte comprises the steps of:
(a) providing a one-way electrolyte inserting valve aid cover, said valve adapted to provide one-way electrolyte access to the inside of the electrolytic chamber; and
(b) at least partially filling said electrolytic chamber with an electrolyte via said valve.

95. A method according to claim 88 including the additional step of filling said liquid chamber with a liquid.

96. A method according to claim 95 wherein said step of filling said liquid chamber with a liquid comprises the steps of:
(a) providing a one-way liquid inserting valve in said base, said valve adapted to provide one way liquid access to the inside of the liquid chamber; and
(b) filling said liquid chamber with a liquid via said valve.

97. A method according to claim 95 wherein said step of filling said liquid chamber with a liquid comprises the steps of:
(a) providing a liquid inserting hole in said base;
(b) filling said liquid chamber with a liquid via said hole; and
(c) sealing said hole thereby sealing said liquid in said liquid chamber.
